(19) United States
(12) Patent Application Publication
(10) Pub. No.: US 2023/0093570 A1
(43) Pub. Date: Mar. 23, 2023

(54) SOLID PREPARATION

(71) Applicants: Sumitomo Pharma Co., Ltd., Osaka (JP); SCREEN HOLDINGS CO., LTD., Kyoto (JP)

(72) Inventors: Yohei NISHIDA, Osaka (JP); Soichiro MORIKAWA, Kyoto (JP)

(21) Appl. No.: 17/801,440

(22) PCT Filed: Feb. 5, 2021
(86) PCT No.: PCT/JP2021/004417

§ 371 Date: Aug. 22, 2022

(30) Foreign Application Priority Data
Feb. 28, 2020 (JP) 2020-033301

(51) Int. Cl. A61K 9/24

(52) U.S. Cl. CPC A61K 9/209

(57) ABSTRACT

Provided is a solid preparation comprising a controlled-release film, wherein the controlled-release film includes a dry film of an edible ink, and contains a water-insoluble polymer, and has excellent controlled-releasability of an active ingredient contained in a tablet core. The solid preparation according to the present invention comprises a tablet core, and at least one layer of a controlled-release film provided on at least a part of a surface of the tablet core, wherein the tablet core contains at least one active ingredient, and the controlled-release film includes a dry film of an edible ink containing at least one water insoluble polymer.

### TECHNICAL FIELD

The present invention relates to a solid preparation, and more specifically relates to a solid preparation in which the release of an active ingredient contained in the inside thereof can be controlled by coating at least a part of a surface of the solid preparation with an edible ink.

### BACKGROUND ART

The surfaces of granules, capsules, tablets, and the like are commonly coated with a coating agent to provide functionalities such as the controlled release of a medicinal component, moisture- and photo-induced degradation of a pharmaceutical agent, masking of smell and bitterness, surface modification, or improved appearance. Among these, the controlled release of a medicinal component can control a supply rate in a manner that the medicinal component is supplied to the human body in an appropriate concentration and rate (time), thereby achieving, for example, a stabilized drug concentration in the blood over a long period of time, a reduced frequency of medication as well as an improved efficacy and stability of pharmacological effects on the human body, and the like.

Methods of coating a surface of a tablet and the like with a coating agent include, for example, a pan coating method comprising spraying a coating liquid having a coating agent dissolved or suspended therein over a tablet and the like, and then allowing the liquid to evaporate (for example, Patent Document 2). In that case, the coating agent may be selected from those coating materials having a solubility in water and the like suitable for the controlled release of a medicinal component and capable of preventing excessive aggregation of tablets during coating. However, in conventional coating technologies in which the pan method is used, application is limited to those having a tablet-like shape which facilitates tablet rolling, for example, round tablets, (R-form, double R-form), couplet tablets, or oval tablets. Coating of flat tablets and the like is difficult. Further, in order to suppress variations in the thickness of a film coating layer, the excessive amount of a film coating liquid needs to be applied. This may cause the following problems: the thickness of a coating is difficult to be precisely controlled, and the releasability of a medicinal component is also difficult to be controlled with good precision. It is noted that Patent Document 1 discloses an edible ink jet composition which enables printing on an orally-disintegrable food product by an inkjet method. However, the edible ink jet composition is intended for controlling neither the disintegrability of a food product in the oral cavity or the digestive tract nor the releasability of a content of a food product.

In the light of the above, a technology about a controlled-release film which enables precisely controlled release of an active ingredient from a tablet, regardless of the tablet shape, has not been yet known practically.

### PRIOR ART DOCUMENTS

### Patent Documents

Patent Document 1: Japanese Patent Application Laid-Open No. 2011-236279
Patent Document 2: Japanese Patent Application Laid-Open No. 2015-3904

### SUMMARY OF THE INVENTION

### Problems to be Solved by the Invention

The present invention is made in view of the aforementioned problems. An object of the present invention is to provide a solid preparation comprising a controlled-release film, wherein the controlled-release film includes a dry film of an edible ink, and contains a water-insoluble polymer, and has excellent controlled-releasability of an active ingredient contained in a tablet core.

### Solutions to the Problems

In order to solve the above problems, the solid preparation according to the present invention comprises a tablet core, and at least one layer of a controlled-release film provided on at least a part of a surface of the tablet core, wherein the tablet core contains at least one active ingredient, and the controlled-release film includes a dry film of an edible ink containing at least one water insoluble polymer.

According to the above configuration, the at least one layer of a controlled-release film is provided on at least a part of the surface of the tablet core. Further, as the controlled-release film, used is one including a dry film of an edible ink and containing a water-insoluble polymer. Then use of an edible ink as a material for a controlled-release film enables the controlled-release film to be formed, for example, by printing by an inkjet method, a dispenser method, or the like. Printing by an inkjet method and the like enables, for example, lamination of multiple layers of controlled-release films and formation of variously patterned controlled-release films. These can provide a solid preparation including a controlled-release film capable of the controlled-release of an active ingredient depending on the structure of a lamination layer and the shape of patterning.

In the above structure, the at least one layer of a controlled-release film may be a patterning layer. A controlled-release film as a patterning layer can further improve the controlled-releasability of an active ingredient as compared to a conventional coating agent in which the controlled release of an active ingredient relies on material selection.

Further, patterning of the controlled-release film may be different for each of any given regions in a plane. This enables different controlled-releasabilities of an active ingredient for each of any given regions.

Furthermore, in the above configuration, the at least one layer of a controlled-release film may have pores for releasing the active ingredient to an outside. Provision of pores in the controlled-release film can prevent an active ingredient being released all at once as the controlled-release film is dissolved. This can control the release of the active ingredient so that the release continues for a certain period of time.

Further, in the above configuration, the total opening area of the pore is in a range of 0.2% to 50% relative to the surface area of the tablet core.

Moreover, in the above configuration, the thickness of the at least one layer of a controlled-release film may be different in a plane. A controlled-release film having different thicknesses in a plane can show different release rates of an active ingredient in the plane. For example, the release of the active ingredient can be reduced at a region having a larger thickness while it can be accelerated at a region having a smaller thickness.

Furthermore, in the above configuration, a structure in which a plurality of the controlled-release films are laminated may be used. Therefore, adjusting the number of lamination layers can further improve the controlled-releasability of the active ingredient.

In the above configuration, the thickness of the controlled-release film preferably is in a range of 0.1 μm to 50 μm. This can maintain the controlled-releasability of the active ingredient and a good mechanical strength of the solid preparation, and can also prevent the solubility, dissolution properties, and disintegration properties of the solid preparation from being impaired due to an excessively enlarged thickness of the controlled-release film.

In the above configuration, the controlled-release film preferably contains one or two or more the water-insoluble polymers each having a solubility of 10 g/100 g or less in water or in an aqueous solution having a pH in the range of 1.2 to 8 at 25° C. This can prevent or reduce the premature dissolution of the controlled-release film including a dry film of an edible ink containing a water-insoluble polymer in water or an aqueous solution having a pH in the range of 1.2 to 8, and thus can prevent or reduce the release of the active ingredient during an unwanted period.

In the above configuration, the water-insoluble polymer is preferably at least one selected from the group consisting of stomach-soluble polymers, enteric polymers, and other water-insoluble polymers.

Further, in the above configuration, the shape of the tablet core may be of a flat tablet or a splitting tablet.

In the above configuration, the active ingredient may be thermally unstable.

Further, in the above configuration, the controlled-release film may include a dry film of the edible ink having a viscosity in a range of 1 mPa·s to 1,010,000 mPa·s.

Moreover, in the above configuration, the controlled-release film preferably controls the release of an active ingredient contained in the tablet core.

### Effects of the Invention

According to the present invention, the controlled-release film for controlling the release of an active ingredient contained in a tablet core includes a dry film of an edible ink and includes a water-insoluble polymer. Therefore, the controlled-release film may be applied by printing according to an inkjet method, a dispenser method, or the like. This can provide a solid preparation having a controlled-release film excellent in the controlled-releasability of an active ingredient as compared to conventional coated tablets in which the controlled release of an active ingredient relies on material selection.

### BRIEF DESCRIPTION OF THE DRAWINGS

FIG. 1 shows a top view of a controlled-release film having a lattice pattern shape in a solid preparation according to an embodiment of the invention.

FIG. 2 shows a top view of a controlled-release film having a stripe pattern shape in a solid preparation according to an embodiment of the invention.

FIG. 3 shows a top view of a controlled-release film having a concentric pattern shape in a solid preparation according to an embodiment of the invention.

FIG. 4 shows a top view of a controlled-release film having a plurality of circular pores in a solid preparation according to an embodiment of the invention.

### EMBODIMENT OF THE INVENTION

(Solid preparation)

A solid preparation according to one embodiment of the present invention will be described below.

The solid preparation according to the present embodiment has a tablet core and at least one layer of a controlled-release film provided on at least a part of a surface of the tablet core.

The term “solid preparation” as used in the present specification is intended to include a food preparation and a pharmaceutical preparation. Examples of food preparations include, for example, health foods such as tablet sweets and supplements. Examples of pharmaceutical preparations include, for example, tablets (film-coated tablets, enteric tablets, sustained-release tablets, oral tablets (sublingual tablets, buccal tablets, and the like)) and the like. Further, there is no particular limitation for the term “tablet core” as used in the present specification as long as it is a solid material which can be taken orally. For example, uncoated tablets, orally-disintegrable tablets (OD tablets), and the like in which raw materials are tableted but left uncoated, may be used as the tablet core.

There is no particular limitation for the overall shape and size of the tablet core, and any of them can be used. Examples of the overall shape of the tablet core include round tablets (R-form, double R-form), couplet tablets, oval tablets, splitting tablets, and the like. Flat tablets and highly friable tablets, which are difficult to be coated by conventional coating technologies, may also be applied. Here, the term “splitting tablet” means a disc-shaped uncoated tablet having a top surface and an under surface, wherein a dividing line of at least one grove is provided on the top surface of the tablet to help divide the tablet, and the top surface is gradually concaved toward the dividing line from the opposite edges, and the under surface of the uncoated tablet is gradually conveyed toward the central region from the peripheral regions, and each of the concave top surface and the convex under surface forms a curved surface so that the central region is thinner than the peripheral regions by having a curvature radius of the top surface smaller than that of the under surface; and the like. “Flat tablets” include, for example, rounded-corner planar tablets, angled-corner planar tablets, and the like. The term “highly friable tablet” means a tablet having a maximum average mass depletion of more than 1.0% as measured by, for example, a method of testing the friability of a tablet in accordance with the Japanese pharmacopoeia, 17th edition; and the like.

The controlled-release film includes a dry film of an edible ink (which will be described in detail below), and contains a water-insoluble polymer (which will be described in detail below). In addition, it has a function of controlling the release of an active ingredient contained inside of a solid preparation to the outside. Further, the controlled-release film may also serve to protect the inside of the solid preparation to increase mechanical strength.

It is noted that the term “active ingredient” as used in the present specification means a medicinal component (a pharmaceutically acceptable active agent) in a pharmaceutical product used for treatment, prevention, diagnosis, and the like of human and animal diseases; a nutrient component in food products and health foods; and the like. Among these, “thermally unstable active ingredients” include, for example, enzymes, peptides, hormones, proteins, ascorbic acid, and the like.

Moreover, the phrase “controlling the release of” as used in the present specification means that the release rate of the solid preparation is slowed as compared to the case for a tablet core having no controlled-release film. For example, the dissolution ratio of a solid preparation to that of a tablet core is 85% or less, preferably 70% or less, even more preferably 50% or less at any of the following time points: 15, 30, 60, 120 minutes when the dissolution rate of an active ingredient is tested in water or in an aqueous solution having a pH in the range of 1.2 to 8 at 37° C. in accordance with a method described in 1st Fluid for Dissolution Test in General Tests of the Japanese Pharmacopoeia (the basket method, the rotation speed: 100 rpm, the volume of a test medium: 900 mL) or a method described in 2nd Fluid for Dissolution Test in General Tests of the Japanese Pharmacopoeia (the paddle method, the rotation speed of paddles: 50 rpm, the volume of a test medium: 900 mL).

The controlled-release film may be provided on at least a part of a surface of a tablet core as described above. For example, in an aspect, the controlled-release film may be provided only on a region (part) of the surface of a tablet core, where the active ingredient is contained. Further, the controlled-release film may be provided so that the entire surface of a tablet core is covered.

Moreover, in addition to the case where the controlled-release film is a flat layer having a uniform thickness in a plane, it may be a patterning layer having various patternings. In the case of the former, the controlled-release film may include, for example, flat layers having thicknesses different at between the front and back surface of a tablet core. Further, in the case of the latter, the controlled-release film may have different patternings for each region in a plane.

When a controlled-release film is a flat layer having a uniform thickness, adjusting the thickness enables the controlled-release of an active ingredient. That is, the release of an active ingredient can be suppressed until the controlled-release film dissolves, thereby creating a time difference between administration and the start of releasing the active ingredient. This allows the release of an active ingredient in a time zone such as night time during which dosing is difficult. Further, this can also avoid adverse effects on the human body due to the interaction of an active ingredient with a combination drug. In particular, the thickness of the controlled-release film can be controlled more precisely by applying the technology of the present invention as compared to the conventional pan coating technology. This enables more precise control over the time of administration and the time of starting the release of an active ingredient.

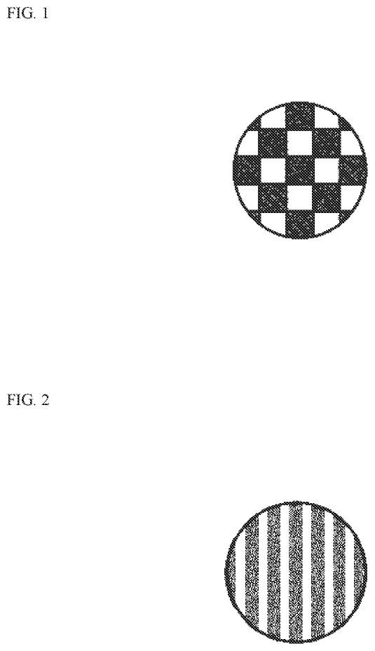

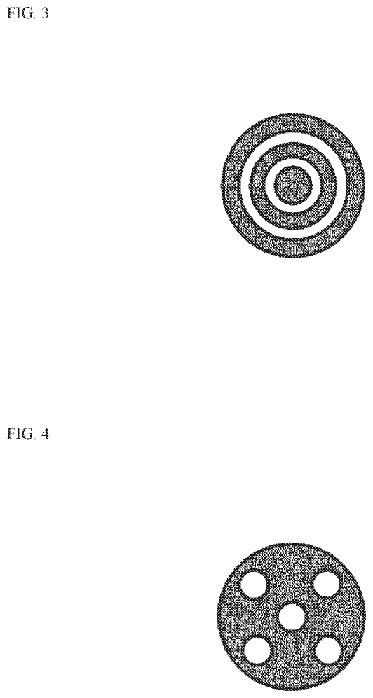

When the controlled-release film is a patterning layer, properly selecting the shape of a patterning enables the controlled-release of an active ingredient. Here, the term “patterning” means forming the shape of a geometrically regular pattern of the controlled-release film, for example, forming two or more regions where the controlled-releasabilities are different from each other due to partial or local differences in thickness, the presence or absence of the film, or the presence or absence, the number, or the shape of pore, and the like, or means the controlled-release film has two or more such regions. Further, a region having the identical controlled-releasability does not need to be mutually continuous, but may be distributed in the manner of a pattern. The term “patterning layer” means a coating having such a patterning applied. As shown in FIGS. 1 to 4, respectively, specific examples of a patterning layer include, for example, a controlled-release film having a lattice-like pattern shape, a controlled-release film having a stripe-like pattern shape, a controlled-release film having a concentric pattern shape, and a controlled-release film having circular pores arranged in equal distance to each other. In each of the pattern shapes shown in FIGS. 1 to 4, a controlled-release film is formed in shaded regions while a tablet core is exposed in the other regions. However, a pattern shape can be formed due to not only the presence or absence of a controlled-release film but also varied thickness.

In the case of the controlled-release film having pores, the surface of a tablet core is exposed at the pores. Therefore, an active ingredient is released to the outside only through the pores until the controlled-release film dissolves. This can prevent the active ingredient from being released all at once, and provide the solid preparation with sustained-releasability. As a result, when the solid preparation is, for example, a pharmaceutical preparation, the blood concentration of an active (medicinal) ingredient can be maintained for a long time. This ensures the persistence of a medicinal ingredient and the safety on the human body.

There is no particular limitation for the alignment of the pores, but it may be, for example, a matrix-like alignment. There is also no particular limitation for the distribution of the pores, but they are preferably uniformly distributed in a plane. This allows for the release amount of an active ingredient to be uniform in a plane of the controlled-release film.

There is no particular limitation for the top view of the pores, but examples include a circular shape, a substantially circular shape, a quadrangular shape, a triangular shape, a lozenge shape, and the like. There is also no particular limitation for the pore size (the area of opening) or the number per unit area (density), and they can be appropriately selected according to the release amount of an active ingredient released through the pores. However, the lower limit of the total area of opening for the pores may be 0.2% or more, 0.4% or more, 0.6% or more, 0.8% or more, 1.0% or more, 2.0% or more, 5.0% or more, 10.0% or more, 15.0% or more relative to the surface area of the tablet core, and the upper limit of the total area of opening for the pores may be 50.0% or less, 45.0% or less, 40.0% or less, 35.0% or less, 30.0% or less, 25.0% or less, 20.0% or less relative to the surface area of the tablet core. The total area of opening for the pores is preferably 0.2% to 50.0%, more preferably 0.2% to 30.0%, and even more preferably 0.4% to 30.0% relative to the surface area of the tablet core. A larger area of opening and a larger density per unit area for the pores can increase the release amount of an active ingredient.

There is no particular limitation for the water-insoluble polymer contained in the controlled-release film, but examples include stomach-soluble polymers, enteric polymers, and other water-insoluble polymers.

There is no particular limitation for the stomach-soluble polymers, but examples include amino acetal compounds such as polyvinylacetal diethylaminoacetate, aminoalkyl methacrylate copolymers E, and mixtures thereof, and the like.

There is no particular limitation for the enteric polymers, but examples include enteric cellulose esters such as cellulose acetate propionate, hydroxypropylmethylcellulose acetate succinate, hydroxypropylmethylcellulose phthalate, hydroxymethylethylcellulose phthalate, carboxymethylethylcellulose, cellulose acetate phthalate: enteric acrylic acid-based copolymers such as methacrylic acid copolymer LD, methacrylic acid copolymer L, methacrylic acid copolymer S: and the like.

There is no particular limitation for the other water-insoluble polymers, but examples include water-insoluble cellulose ethers such as ethyl cellulose, ethyl methyl cellulose, ethyl propyl cellulose, isopropyl cellulose, butyl cellulose, benzyl cellulose, cyanoethyl cellulose; water-insoluble acrylic acid-based copolymers such as ethyl acrylate-methyl methacrylate trimethylammonium ethyl chloride methacrylate copolymer, ethyl acrylate-methyl methacrylate copolymer; and the like.

Among the stomach-soluble polymers, the enteric polymers, and the other water-insoluble polymers exemplified above, preferred are water-insoluble polymers which are insoluble across the entire range of pH (that is, a pH in the range of 0 to 14) and water-insoluble polymers which are insoluble outside a certain range of pH (for example, a pH in the range of 5 to 14). Further, among the water-insoluble polymers which are insoluble across the entire range of pH and the water-insoluble polymers which are insoluble outside a certain range of pH, enteric polymers and other water-insoluble polymers are more preferred, and enteric polymers are most preferred.

A choice of a water-insoluble polymer contained in a controlled-release film enables the controlled-release of an active ingredient from a solid preparation. For example, when an enteric polymer is contained as a water-insoluble polymer, and the controlled-release film according to the present invention is used as an enteric controlled-release film, the release of an active ingredient from the solid preparation can be suppressed within the stomach, and the degradation of a drug liable to gastric acid can be avoided, and the time of releasing an active ingredient can be controlled. Furthermore, when a stomach-soluble polymer is contained as a water-insoluble polymer, and the controlled-release film according to the present invention is used as a stomach-soluble controlled-release film, the release of an active ingredient from a tablet within the oral cavity can be suppressed, leading to reduced bitterness of a drug.

The solubility of a water-insoluble polymer contained in a controlled-release film is preferably 10 g/100 g or less, more preferably 5 g/100 g or less in water or in an aqueous solution having a pH in the range of 1.2 to 8 at 25° C. When a water-insoluble polymer contained in a controlled-release film has a high solubility in water or an aqueous solution having a pH in the range of 1.2 to 8, the controlled-release film dissolves immediately in a solution into which the controlled-release is intended, resulting in uncontrolled release of an active ingredient even if the thickness of the controlled-release film is increased. However, when a water-insoluble polymer contained in a controlled-release film has a solubility of 10 g/100 g or less in water or an aqueous solution having a pH in the range of 1.2 to 8, the release of an active ingredient at unwanted times can be prevented or reduced. Further, suppression of the readily dissolution of the controlled-release film allows an active ingredient to be released in a controlled manner by adjusting the thickness of the controlled-release film.

There is no particular limitation for the aqueous solution having a pH in the range of 1.2 to 8, but examples include a solution (pH 1.2) described in 1st Fluid for Dissolution Test and a solution (pH 6.8) described in 2nd Fluid for Dissolution Test in accordance with the Japanese pharmacopoeia, 17th edition.

There is no particular limitation for the thickness of a controlled-release film, and it can be appropriately selected depending on the material of the controlled-release film, the solubility of a water-insoluble polymer contained in the controlled-release film in water or an aqueous solution having a pH in the range of 1.2 to 8, the release amount of an active ingredient, and the like. Usually, the thickness of a controlled-release film is in a range of 0.1 μm to 50 μm, preferably 0.5 μm to 20 μm, more preferably 1 μm to 10 μm. A thickness of a controlled-release film of 0.1 μm or more can maintain the controlled-releasability of an active ingredient. Further, the mechanical strength of a solid preparation can also be well maintained. On the other hand, a thickness of a controlled-release film of 50 μm or less can prevent the solubility, dissolution properties, and disintegration properties of a solid preparation from being imparted due to the excessively enlarged thickness of the controlled-release film. Therefore, they can be well maintained. It is noted that the thickness of a controlled-release film may be appropriately selected within the aforementioned numerical range when it is a patterning layer.

Moreover, a plurality of controlled-release films may be laminated into a multilayer structure. In this case, the same type of controlled-release films may be used for lamination, or the multiple types of controlled-release films may be used for lamination. The release rate of an active ingredient can be controlled by appropriately selecting the type and the number of lamination layers of a controlled-release film. Moreover, the mechanical strength of a solid preparation can also be controlled by appropriately selecting the number of lamination layers of a controlled-release film. Specifically, the mechanical strength of a solid preparation can be increased by increasing the number of lamination layers as described above.

In the case of a multilayer structure in which a plurality of controlled-release films are laminated, the thickness of the multilayer structure is preferably within the range of 0.1 μm to 50 μm, more preferably within the range of 0.5 μm to 20 μm, and in particular preferably within the range of 1 μm to 10 μm.

The controlled-release film according the present embodiment includes a dry film of an edible ink as described above. There is no particular limitation for the edible ink as long as it can be orally administered. This can make the controlled-release film edible. Specific examples include materials in accordance with pharmaceutical additives listed in the Pharmaceutical Affairs Law, the Japanese pharmacopoeia, or the Japanese Standards of Food Additives.

Below, the case where non-aqueous ink is used as an edible ink will be described as an example. The term “non-aqueous ink” as used in the present specification means that the non-aqueous ink does not contain water or contains a small quantity of water. More specifically, the phrase “contains a small quantity of” means that 30 mass % or less of water, preferably 20 mass % or less of water, more preferably 10 mass % of water is contained on the basis of mass of a non-aqueous ink.

The non-aqueous ink is preferably a non-aqueous ink for inkjet when a controlled-release film is printed and formed by an inkjet method as described below. However, the non-aqueous ink for inkjet may also be adapted to printing by a dispenser method as described below.

The non-aqueous ink for inkjet contains at least a non-aqueous ink composition for inkjet (hereinafter referred to as a “non-aqueous ink composition”), and enables the formation of a controlled-release film on at least a part of a surface by printing with the non-aqueous ink for inkjet. It is noted that in the present embodiment, the non-aqueous ink for inkjet may be a non-aqueous ink composition itself, otherwise it may contain the non-aqueous ink composition.

The non-aqueous ink composition contains at least an edible water-insoluble polymer and a solvent as the main solvent. The water-insoluble polymer is preferably present in a state of being dissolved in a solvent.

The non-aqueous ink composition may have optical transparency in the region of visible light (400 nm to 760 nm). Further, the optical transparency of the non-aqueous ink composition may be colorless transparent or may be chromatic transparent. Use of a non-aqueous ink composition having optical transparency enables the formation of a controlled-release film having optical transparency. Thereby, a controlled-release film can also be formed on an ink layer which forms a printing image such as a character without preventing visibility. It is noted that the term “optical transparency” as used in the present specification means a property which transmits at least a part of incident visible light. More specifically, the transmittance of visible light in a wavelength region of 400 nm to 800 nm through a controlled-release film having a thickness of 2 μm is higher by 50% or more, more preferably 70% or more, even more preferably 90% or more than that in the absence of the controlled-release film (in the case where the transmittance is 100%).

The viscosity of a non-aqueous ink composition is preferably 1 mPa to 1,000,000 mPa·s for printing by a dispenser method. Further, for printing with an inkjet method, the viscosity is preferably 1 mPa·s to 100 mPa·s at the time of being discharged through a nozzle, and more preferably 3 mPa·s to 12 mPa·s, even more preferably 3 mPa·s to 10 mPa·s, and in particular preferably 3 mPa·s to 8 mPa·s considering the discharge stability through a nozzle of an inkjet head. When the viscosity of a non-aqueous ink composition is in the above numerical range, the development of clogging at a nozzle of an inkjet head can be suppressed to maintain good discharge stability, which can, in turn, prevent reduced flying properties. It is noted that the viscosity of a non-aqueous ink composition can be determined by measurement with, for example, an oscillating viscosimeter (Product name; VISCOMATE MODEL VM-10A, available from Sekonic Corporation) at a measurement temperature of 25° C.

The solubility of a water-insoluble polymer contained in a non-aqueous ink is as described above. Therefore, details thereof are omitted.

The concentration of the solid content in a water-insoluble polymer is preferably in the range of 5 mass % to 30 mass %, more preferably in the range of 7.5 mass % to 25 mass %, and in particular preferably in the range of 10 mass % to 20 mass % relative to the total mass of the non-aqueous ink composition. When the concentration of the solid content in a water-insoluble polymer is 5 mass % or more, the controlled-releasability of an active ingredient can be well maintained. On the other hand, a concentration of the solid content in a water-insoluble polymer of 30 mass % or less can prevent deterioration of the solubility, dissolution properties, and disintegration properties of a solid preparation due to an excessively enlarged thickness of the controlled-release film, and can maintain them in good conditions. This can also prevent reduced discharge performance due to an excessively large viscosity of the non-aqueous ink composition.

The aforementioned solvent is preferably a non-aqueous solvent (or an organic solvent). A tablet core shows solubility into water. Therefore, when a non-aqueous solvent is used as a solvent, the solvent can prevent dissolution of the outermost surface layer of the tablet core.

There is no particular limitation for the non-aqueous solvent as long as it is not miscible with water, but examples include at least one selected from the group consisting of propylene glycol, glycerin, ethanol, butanol, isobutanol, propanol, isopropanol, pentyl alcohol, ethyl lactate, ethyl acetate, triethyl citrate, and acetone.

Other additives may also be blended in the above non-aqueous ink composition. Other additives include surface tension modifiers, wetting agents, organic amines, surfactants, pH adjusters, chelating agents, antiseptic agents, viscosity modifiers, antifoaming agents, plasticizing agents, coloring agents, and the like. The non-aqueous ink composition according to the present embodiment is used for printing on a solid preparation such as food preparation and pharmaceutical preparation. Therefore, these other additives are preferably materials in accordance with pharmaceutical additives listed in the Pharmaceutical Affairs Law, the Japanese pharmacopoeia, or the Japanese Standards of Food Additives.

There is no particular limitation for surface tension modifiers, but specific examples include glycerin fatty acid esters, polyglycerin fatty acid esters, and the like. Glycerin fatty acid esters include, for example, decaglyceryl caprylate, hexaglycerol laurate ester, hexaglycerol oleate ester, condensed tetraglycerol linolenate ester, palm fatty acid ester, decaglyceryl laurate having an HLB of 15 or less, decaglyceryl oleate having an HLB of less than 13, and the like. Further, polyglycerin fatty acid esters include polyglyceryl monostearate, polyglyceryl monoleate, polyglyceryl monoisostearate, polyglyceryl monolaurate, condensed polyglyceryl ricinoleate, decaglyceryl monomyristate, polyglyceryl pentaisostearate, polyglyceryl pentaoleinate, polyglyceryl heptastearate, polyglyceryl decaoleinate, and the like. These may be used alone or in a mixture of two or more.

The content of a surface tension modifier is preferably in the range of 0.1 mass % to 5 mass %, more preferably in the range of 1 mass % to 2 mass % relative to the total mass of the non-aqueous ink composition. A content of a surface tension modifier of 0.1 mass % or more can prevent deteriorated discharge due to poor formulation of meniscus at a nozzle of an inkjet head when printing by an ink jet method, which can, in turn, prevent the development of clogging of the nozzle. As a result, discharge stability can be improved. On the other hand, a content of a surface tension modifier of 5 mass % or less can prevent adverse effects on discharge due to insoluble matter or poor emulsification of the surface tension modifier.

There is no particular limitation for wetting agents, but they include those satisfying the criteria in accordance with the Pharmaceutical Affairs Law, and the like. Specific examples include polyethylene glycol, propylene glycol, glycerin, and the like.

The addition amount of a wetting agent is preferably 1 mass % to 50 mass %, more preferably 10 mass % to 40 mass % relative to the total mass of a non-aqueous ink composition. The content of a wetting agent of 1 mass % or more can prevent clogging in the vicinity of a nozzle of an inkjet head when printing by an inkjet method to further improve discharge performance. On the other hand, a content of a wetting agent of 50 mass % or less can control the viscosity of a non-aqueous ink composition in an appropriate manner.

It is noted that there is no particular limitation for the content of an additive such as organic amine, a surfactant, a pH adjuster, a chelating agent, an antiseptic agent, a viscosity modifier, and an antifoaming agent in the corresponding non-aqueous ink composition, and it may be selected appropriately as needed.

The above non-aqueous ink composition may be produced by mixing the aforementioned components by a proper method. There is no particular limitation for the method of mixing and the order of addition. After mixing, sufficient stirring is performed, and filtration is then performed, if required, to remove coarse particles and foreign matters which may cause clogging. Thereby, the non-aqueous ink composition according to the present embodiment can be obtained.

There is no particular limitation for the method of mixing those materials, but stirring and mixing can be achieved by adding materials sequentially to a container equipped with a stirring mechanism such as a disper, a mechanical stirrer, and a magnetic stirrer. There is also no particular limitation for the method of filtration, but, for example, centrifugal filtration, filter filtration, and the like can be used.

As described above, the solid preparation according to the present embodiment has a configuration in which at least one layer of a controlled-release film is provided on at least a part of a surface thereof. The controlled-release film is intended for the controlled-release of an active ingredient by taking advantages of the thickness and laminate structure thereof, various patterning shapes. In this respect, it has superior controlled-release film as compared to conventional coating agents in which the controlled-release of an active ingredient relies on material selection.

### (Method of Producing Solid Preparation)

A method of producing a solid preparation includes a step of forming at least one layer of a controlled-release film on a part of a surface of a tablet core, in which an active ingredient is to be released across that part.

The step of forming a controlled-release film can be performed, for example, by a printing method such as an inkjet method and a dispenser method when a non-aqueous ink is used as an edible ink.

When printing is performed by the inkjet method, a non-aqueous ink for inkjet is preferably used which contains the aforementioned non-aqueous ink composition as a non-aqueous ink. Printing by the inkjet method may be performed by discharging a non-aqueous ink for inkjet as droplets through a fine nozzle to allow the droplets to adhere on at least a part of a surface of a tablet core. Printing by the inkjet method can also be achieved by a so-called one path (single path) printing or a so-called shuttle (scan) printing. In the so-called one pass (single pass) printing, discharge ports for a non-aqueous ink for inkjet are aligned in the direction orthogonal to the direction for scanning a tablet core, and the formation of a controlled-release film is completed by allowing the tablet core to pass through under the discharge ports only once. In the so-called shuttle (scan) printing, a controlled-release film is formed on a surface of a tablet core by performing a main scan with discharge ports on the tablet core in a scan direction (straight scan direction), and intermittently performing a sub-scan in the width direction after each completion of the main scan. In the former single pass printing, a controlled-release film can be printed at high speed to improve productivity. Further in the latter shuttle (scan) printing, a controlled-release film having high definition patterning applied can be formed to further improve the accuracy of controlled-release.

Furthermore, there is no particular limitation for the method of discharging a non-aqueous ink for inkjet, but known methods may be used such as a continuous jetting type (a charge control type, a spray type, and the like), an on-demand type (a piezo method, a thermal method, an electrostatic aspiration method, and the like), for example. There is also no particular limitation for the conditions of discharging a non-aqueous ink for inkjet, and they are selected appropriately.

Moreover, when printing is performed by the aforementioned dispenser method, an appropriate amount of a non-aqueous ink is discharged under control of air pressure and discharge time to allowing the non-aqueous ink to adhere on at least a part of a surface of a tablet core. In the case of the dispenser method, a non-aqueous ink having a higher viscosity as compared to the inkjet method can be used. Further, in the dispenser method, a larger amount of a non-aqueous ink can be discharged than in the inkjet method, and thus a controlled-release film having a larger thickness can be formed.

The application amount (the discharge amount, the droplet amount, or the adhesive amount) of a non-aqueous ink is preferably 0.1 mg/cm² to 50 mg/cm², more preferably 0.5 mg/cm² to 20 mg/cm², further preferably 1 mg/cm² to 10 mg/cm². An application amount of a non-aqueous ink of 0.1 mg/cm² or more can prevent the controlled-releasability of a controlled-release film from being excessively lowered. The mechanical strength of a solid preparation can also be well maintained. On the other hand, an application amount of a non-aqueous ink of 50 mg/cm² or less can prevent excessively impaired release of an active ingredient due to an excessively enlarged thickness of the controlled-release film. Therefore, a good controlled-releasability can be maintained. Further, a drying time after application of a non-aqueous ink can also be controlled.

In the step of forming a controlled-release film, a drying process may also be performed on a coating of a non-aqueous ink adhering on a surface of a tablet core. The drying process can be performed, for example, after printing on at least a part of a surface of a tablet core. There is no particular limitation for the method of drying, but natural drying, hot-air drying, or the like can be performed. There is also no particular limitation for drying conditions such as drying time and drying temperature, but they can be selected depending on the application amount of a non-aqueous ink.

In a method of forming a film according to the conventional pan coating method, it is necessary to perform coating after filling a pan volume of a coating apparatus with tablets in an amount equal to or more than a certain proportion as described above. This, disadvantageously, makes it difficult for small-lot production. In contrast, a method of forming a film by the inkjet method according to the present invention, and the like enables small-lot production. Moreover, the present invention enables single-tablet based drying even when an edible ink applied to a surface of a tablet core is dried during formation of a controlled-release film. Therefore, drying may be done in a shorter time as compared to the conventional pan coating method, and coating may also be performed on a tablet containing a thermally unstable active ingredient, and the like.

### (Other Considerations)

In the present embodiment, examples where a non-aqueous ink is used as an edible ink are used to describe the present invention. However, the present invention shall not be limited to this aspect. An aqueous ink may be used as an edible ink. The term “aqueous ink” as used in the present specification means an ink in which an aqueous medium is used as a solvent.

An aqueous ink contains at least an aqueous ink composition for inkjet (hereinafter referred to an “aqueous ink composition”). Aqueous ink compositions include, for example, those composed of a dispersion in which the aforementioned water-insoluble polymer is nano-dispersed in an aqueous medium. Aqueous media include water and a solvent mixture of water and a water-soluble organic solvent. There is no particular limitation for the types of water-soluble organic solvents as long as they are edible. It is noted that nano-dispersion means that a polymer component is dispersed in an aqueous medium alone or in a state of a fine particle having a size in the order of nanometer or less including the water-insoluble polymer. Further, an aqueous ink may be an aqueous ink composition itself, otherwise it may include the aqueous ink composition.

The 50% particle size (D50) in terms of the cumulative particle size in the volume-based cumulative particle size distribution of a water-insoluble polymer dispersed in an aqueous medium is preferably 10 nm to 1000 nm, more preferably 50 nm to 500 nm. A D50 of 10 nm or more can prevent deterioration of the dispersion stability and discharge stability of a water-insoluble polymer. On the other hand, a D50 of 1000 nm or less can prevent separation or sedimentation of a water-insoluble polymer, allowing the dispersion stability to be maintained. Further the 99% particle size (D99) in terms of the cumulative particle size in the volume-based cumulative particle size distribution of a water-insoluble polymer is preferably 2000 nm or less, more preferably 1000 nm or less.

Considering the discharge stability through a nozzle of an inkjet head, the viscosity of an aqueous ink composition is preferably 3 mPa·s to 12 mPa·s, more preferably 3 mPa·s to 10 mPa·s, and in particular preferably 3 mPa·s to 8 mPa·s at the time of being discharged through the nozzle. When the viscosity of an aqueous ink composition is in the above numerical range, the development of clogging at a nozzle of an inkjet head can be suppressed to maintain good discharge stability, which can, in turn, prevent reduced flying properties. It is noted that the viscosity of an aqueous ink composition can be determined by measurement with, for example, an oscillating viscosimeter (Product name: VISCOMATE MODEL VM-10A, available from Sekonic Corporation) at a measurement temperature of 25° C.

In the case where an aqueous ink is used as an edible ink, a controlled-release film can also be formed by printing by an inkjet method, a dispenser method, or the like. The details of the method of forming a controlled-release film using an aqueous ink are similar to those of the case where the aforementioned non-aqueous ink is used.

### EXAMPLES

Below, Examples suitable for the present invention will be described in detail by way of example. However, materials, contents, and the like described in the following

Examples shall not limit the scope of the present invention to them, unless otherwise specifically stated as limitation.

Further, agents and additives used in the following Examples and Comparative Examples were as follows unless otherwise specifically noted.
Haloperidol: Dainippon Sumitomo Pharma Co., Ltd.
Lactose hydrate (Pharmatose 200M): DFE Pharma
Maize starch (Cornstarch XX16): Nihon Shokuhin Kako Co., Ltd.
Carmellose calcium (ECG-505): Gotoku Chemical Co., Ltd.
Polyvinyl alcohol (partially hydrolyzed) (Gosenol EG-05P): Nippon Synthetic Chemical Industry Co., Ltd.
Magnesium stearate (plant origin): Taihei Chemical Industrial Co., Ltd.
Amino alkyl methacrylate copolymer E (Eudragit EPO): Evonik Japan Co., Ltd.
Sodium lauryl sulfate: Spectrum
Stearic acid (powder NF-GenAR): AVANTOR
Talc (high grade talc (MSP)): Nippon Talc Co., Ltd.
Red No. 2 (Food Red No. 2): San-Ei Gen F. F. I., Inc.
Blue No. 1: San-Ei Gen F. F. I., Inc.

### Example 1

First, 10 mass % of amino alkyl methacrylate copolymer E as a water-insoluble polymer was mixed with 90 mass % of ethanol as a solvent according to the mixing composition shown in Table 1 to produce a non-aqueous ink for inkjet. The solubility of this amino alkyl methacrylate copolymer E in water (25° C.) was 10 g/100 g or less. It is noted that both of the materials for the non-aqueous ink are compatible with materials in accordance with pharmaceutical additives listed in the Pharmaceutical Affairs Law, the Japanese pharmacopoeia, or the Japanese Standards of Food Additives.

TABLE 1

| Type of material | Component | Content (mass %) |
|---|---|---|
| Water-insoluble | Amino alkyl methacrylate | 10 |
| polymer | copolymer E | |
| Solvent | Ethanol | 90 |

Next, the non-aqueous ink for inkjet was used to perform printing on a surface of an uncoated tablet (a tablet core) by an inkjet method, thereby forming a film including the non-aqueous ink for inkjet. Printing was performed in accordance with the single pass (one pass) method using an ink jet printer (KC 600 dpi head, a medium speed printing jig). Printing was performed under the environment of an air temperature of 25° C. and a relative humidity of 50%, and the application amount (discharge amount) of the non-aqueous ink for inkjet was 2 mg/cm².

Subsequently, the printed surface was sufficiently dried by natural drying. The drying time was one minute. Thereby, a controlled-release film having a plurality of small pores and including a dry film of the non-aqueous ink for inkjet was formed on a surface of the uncoated tablet.

It is noted that the thickness of the controlled-release film was about 2 μm. Further, the small pores in the controlled-release film were formed as substantially circular and fine pores having a diameter of about 100 μm so as to be aligned in a matrix manner. Furthermore, the number of small pores per unit area was 200/cm².

The solid preparation of this Example obtained as described above, which includes a controlled-release film having fine small pores with a diameter of about 100 μm aligned in a matrix manner on a surface thereof, has superior controlled-releasability as compared to a solid preparation comprising, for example, a conventional coating agent in which the controlled release of an active ingredient relies on material selection.

### Example 2

In this Example, a solid preparation (film coated tablet) without pores containing 1 mg of haloperidol was produced as follows.

First, granulated granules, mixed powder, and uncoated tablets were produced as follows according to the compositions shown in Table 2.

TABLE 2

| | | Formulation | |
|---|---|---|---|
| | Component | (mg) | (wt %) |
| Granule | Haloperidol | 1 | 0.56 |
| component | Lactose hydrate | 159.4 | 88.56 |
| | Maize starch | 8 | 4.44 |
| | Carmellose calcium | 5.4 | 3.00 |
| | Polyvinyl alcohol (partially hydrolyzed) | 5.4 | 3.00 |
| | Subtotal of granulated granules | 179.2 | 99.56 |
| Mixture | Magnesium stearate | 0.8 | 0.44 |
| component | Subtotal of mixed powder and uncoated | 180 | 100 |
| | tablet | | |

### (1) Production of a 10% Haloperidol Powder (1)

In a polyethylene bag, 29.99 g of haloperidol and 270.02 g of lactose hydrate were mixed for 1 minute, and allowed to pass through a size sieving equipment (available from Powrex corp.. Comil model 194S) set at a mesh opening of 0.610 mm and a rotation speed of 1500 rpm to obtain a 10% haloperidol powder (1).

### (2) Preparation of 4 wt % Polyvinyl Alcohol (Partially Hydrolyzed) Solution (2)

Purified water in an amount of 1920.01 g was weighed out, and about one half of it was transferred into a stainless steel beaker, and heated. Then 80.01 g of polyvinyl alcohol (partially hydrolyzed) was slowly added and dissolved with stirring. After allowed to stand for cooling, additional purified water in an amount corresponding to the weight of evaporated purified water was added and stirred again to prepare a 4 wt % polyvinyl alcohol (partially hydrolyzed) solution (2).

(3) Production of granulated granules containing haloperidol

Granulated granules containing haloperidol were produced according to the charge—in quantities shown in Table 3 and the production conditions as described in Table 4. First, the 10% haloperidol powder (1), lactose hydrate, cornstarch, and carmellose calcium were charged into a fluidized bed granulator and dryer (available from Powrex corp., Multiplex model MP-01) according to the charge—in quantities shown in Table 3, and allowed to flow for mixing until the outlet temperature became 38° C. Subsequently, the 4 wt % polyvinyl alcohol (partially hydrolyzed) solution (2) in the charge—in quantity shown in Table 3 was sprayed while flowing. Then, it was allowed to flow for drying until the outlet temperature became 45° C. and then removed from the container. Further, the granules removed were allowed to pass through a screen with a mesh opening of 1000 μm to obtain granulated granules containing haloperidol.

TABLE 3

| | | Charge-in |
|---|---|---|
| | Component | quantity (g) |
| Granule | 10% Haloperidol powder (1) | 60.02 |
| component | Lactose hydrate | 902.39 |
| | Corn starch | 48.01 |
| | Carmellose calcium | 32.40 |
| | 4 wt % polyvinyl alcohol (partially hydrolyzed) | 810.0 |
| | solution (2) | |

TABLE 4

| Items | Production conditions |
|---|---|
| Container size | Standard container |
| Gun diameter | 1.2 mm |
| Gun height | Middle |
| Spray type | Top spray |
| Spray pressure | 0.12 MPa |
| Inlet air | 75° C. |
| temperature | |
| Charge air flow | 65 m³/hr |
| Spray rate | About 10 g/min |

### (4) Production of Mixed Powder Containing Haloperidol

A mixed powder containing haloperidol was produced according to the charge—in quantities shown in Table 5. Into a V10 container, charged were the granulated granules containing haloperidol in the charge—in quantity shown in Table 5 followed by magnesium stearate in the charge—in quantity shown in Table 5, and mixed with a mixer (available from Tsutsui Scientific Instruments Co., Ltd., model S-5) at 40 rpm for 5 minutes.

TABLE 5

| | | Charge-in |
|---|---|---|
| | Component | quantity (g) |
| Mixture | Granulated granules containing haloperidol | 952.11 |
| component | Magnesium stearate | 4.25 |

### (5) Production of Uncoated Tablets Containing 1 mg of Haloperidol

Uncoated tablets were produced by tableting the resulting mixed powder containing haloperidol with a tableting machine (available from Kikusui Seisakusho Ltd., model VELA2) under the conditions described in Table 6 so that 1 mg of haloperidol was contained in one tablet.

TABLE 6

| Items | Production conditions |
|---|---|
| Punch size and | φ 8.0 mm, planar rounded-comer |
| shape | |
| Rotation speed | 30 rpm |
| Main pressure | 10 kN |

### (6) Preparation of Non-Aqueous Ink (3)

According to the blending composition as shown in Table 7, 10 mass % amino alkyl methacrylate copolymer E, 0.5 mass % propylene glycol, 0.1 mass % Blue No. 1, 88.4 mass % ethanol as a solvent, and 1 mass % water were mixed to obtain a non-aqueous ink (3) and (film coating liquid). The solubility of amino alkyl methacrylate copolymer E included in this non-aqueous ink (3) was 10 g/100 g or less in water (25° C.).

TABLE 7

| Component | Content (mass %) |
|---|---|
| Amino alkyl methacrylate | 10 |
| copolymer E | |
| Propylene glycol | 0.5 |
| Blue No. 1 | 0.1 |
| Water | 1 |
| Ethanol | 88.4 |

### (7) Production of Film Coated Tablet Containing 1 mg of Haloperidol

The non-aqueous ink (3) was sprayed uniformly on the under and side surfaces of an uncoated tablet, and allowed to dry under the conditions as described in Table 8. Subsequently, the non-aqueous ink (3) was used to form a controlled-release film having no pores on the top surface of the uncoated tablet by an ink jet method, thereby producing a film coated tablet containing 1 mg of haloperidol.

TABLE 8

| Device used | Airbrush |
|---|---|
| Coating amount of film coating | 1.4 mg/tablet |
| | (Total weight of dry films formed on the |
| | under and side surfaces) |
| Drying | Dried in hot air at 65° C. with a wind |
| | speed of 10 m/s for about 20 seconds. |

Printing on the top surface of the uncoated tablet by an ink jet method was performed in accordance with the single pass (one pass) method using an ink jet printer (KC 600 dpi head, a medium speed printing jig). Further, printing was performed under the environment of an air temperature of 25° C. and a relative humidity of 50%, and application was repeated 10 times on the top surface with an application amount (discharge amount) of the non-aqueous ink (3) of about 1 mg/cm² per single pass.

Then, the printed surface was sufficiently dried with hot air. The drying time was 30 seconds. Thereby, a controlled-release film including a dry film of the non-aqueous ink (3) was formed on the surface of the uncoated tablet. It is noted that the thickness of the controlled-release film was about 4 μm.

### Example 3

In this Example, a solid preparation (film coated tablet) with pores containing 1 mg of haloperidol was produced as follows.

(1) Production of Uncoated Tablet Containing 1 mg of Haloperidol An uncoated tablet containing 1 mg of haloperidol was produced as in Example 2 (refer to (1) to (5) in Example 2).

### (2) Preparation of Non-Aqueous Ink (3)

A non-aqueous ink (3) was prepared as in Example 2 (refer to (6) in Example 2).

(3) Production of Film Coated Tablet with Pores Containing 1 mg of Haloperidol

The non-aqueous ink (3) was spray-coated uniformly on the under and side surfaces of the resulting uncoated tablet, and allowed to dry under the conditions as described in Table 9. Subsequently, the non-aqueous ink (3) was used to perform printing on the top surface of the uncoated tablet by an inkjet method to form a controlled-release film with pores. Thereby, produced was a film coated tablet which contains 1 mg of haloperidol and has drug releasability.

TABLE 9

| Device used | Airbrush |
|---|---|
| Coating amount of film coating | 1.4 mg/tablet |
| | (Total weight of dry films formed |
| | on the under and side surfaces) |
| Drying | Dried in hot air at 65° C. with a wind |
| | speed of 10 m/s for about 20 seconds. |

Printing on the top surface of the uncoated tablet by the ink jet method was performed in accordance with the single pass (one pass) method using an ink jet printer (KC 600 dpi head, a medium speed printing jig). Further, printing was performed under the environment of an air temperature of 25° C. and a relative humidity of 50%, and application was repeated 10 times on the top surface with an application amount (discharge amount) of the non-aqueous ink (3) of about 1 mg/cm² per single pass. After printing, the printed surface was further dried with hot air. The drying time was 30 seconds.

It is noted that the thickness of the controlled-release film was about 4 μm. Moreover, patterning was performed by the ink jet method so that the pores of the controlled-release film have a circular shape with a diameter of 1 mm. Furthermore, the number of pores formed on the top surface of the solid preparation was 1, 3, or 5/tablet for each film coated tablet.

The solid preparations according to this Example obtained as described above each have a controlled-release film having 1, 3, or 5 pores with a diameter of about 1 mm on the surface thereof.

### Comparative Example 1

In this Comparative Example, a film coated tablet was produced which does not have the controlled-release film according to the present invention.

First, granulated granules, mixed powder, and uncoated tablets were produced as follows according to the compositions shown in Table 10.

TABLE 10

| | | Formulation | |
|---|---|---|---|
| | Component | (mg) | (wt %) |
| Granule | Haloperidol | 1 | 0.56 |
| component | Lactose hydrate | 159.4 | 88.56 |
| | Maize starch | 8 | 4.44 |
| | Carmellose calcium | 5.4 | 3.00 |
| | Polyvinyl alcohol (partially hydrolyzed) | 5.4 | 3.00 |
| | Subtotal of granulated granules | 179.2 | 99.56 |
| Mixture | Magnesium stearate | 0.8 | 0.44 |
| component | Subtotal of mixed powder and uncoated | 180 | 100 |
| | tablet | | |

### (1) Production of 10% Haloperidol Powder (1)

In a polyethylene bag, 29.99 g of haloperidol and 270.02 g of lactose hydrate were mixed for 1 minute, and allowed to pass through a size sieving equipment (available from Powrex corp., Comil model 194S) set at a mesh opening of 0.610 mm and a rotation speed of 1500 rpm to obtain a 10% haloperidol powder (1).

### (2) Preparation of 4 wt % Polyvinyl Alcohol (Partially Hydrolyzed) Solution (2)

Purified water in an amount of 1920.01 g was weighed out, and about one half of it was transferred into a stainless steel beaker, and heated. Then 80.01 g of polyvinyl alcohol (partially hydrolyzed) was slowly added and dissolved with stirring. After allowed to stand for cooling, additional purified water in an amount corresponding to the weight of evaporated purified water was added and stirred again to prepare a 4 wt % polyvinyl alcohol (partially hydrolyzed) solution (2).

### (3) Production of Granulated Granules Containing Haloperidol

Granulated granules containing haloperidol were produced according to the charge—in quantities shown in Table 11 and the producing conditions shown in Table 12. First, the 10% haloperidol powder (1), lactose hydrate, cornstarch, and carmellose calcium were charged into a fluidized bed granulator and dryer (available from Powrex corp., Multiplex MP-01) according to the charge—in quantities shown in Table 10, and allowed to flow for mixing until the outlet temperature became 38° C. Subsequently, the 4 wt % polyvinyl alcohol (partially hydrolyzed) solution (2) in the charge—in quantity shown in Table 10 was sprayed while flowing. Then, the granules were allowed to flow for drying until the outlet temperature became 45° C., and then removed from the container. Further, the granules removed were allowed to pass through a screen with a mesh opening of 1000 μm to obtain granulated granules containing haloperidol.

TABLE 11

| | | Charge-in |
|---|---|---|
| | Component | quantity (g) |
| Granule | 10% Haloperidol powder (1) | 60.02 |
| component | Lactose hydrate | 902.39 |
| | Corn starch | 48.01 |
| | Carmellose calcium | 32.40 |
| | 4 wt % polyvinyl alcohol (partially | 810.0 |
| | hydrolyzed) solution (2) | |

TABLE 12

| Items | Production conditions |
|---|---|
| Container size | Standard container |
| Gun diameter | 1.2 mm |
| Gun height | Middle |
| Spray type | Top spray |
| Spray pressure | 0.12 MPa |
| Inlet air | 75° C. |
| temperature | |
| Charge air flow | 65 m³/hr |
| Spray rate | About 10 to 11 g/min |

### (4) Preparation of Mixed Powder Containing Haloperidol

A mixed powder containing haloperidol was produced according to the charge—in quantities shown in Table 13. Into a V10 container, charged were the granulated granules containing haloperidol in the charge—in quantity shown in Table 13 followed by magnesium stearate in the charge—in quantity shown in Table 13, and mixed with a mixer (available from Tsutsui Scientific Instruments Co., Ltd., model S-5) at 40 rpm for 5 minutes.

TABLE 13

| | | Charge-in |
|---|---|---|
| | Component | quantity (g) |
| Mixture | Granulated granules containing haloperidol | 948.58 |
| component | Magnesium stearate | 4.23 |

### (5) Production of Uncoated Tablet Containing 1 mg of Haloperidol

Uncoated tablets were produced by tableting the resulting mixed powder containing haloperidol with a tableting machine (available from Kikusui Seisakusho Ltd., model VELA2) under the conditions described in Table 14 so that 1 mg of haloperidol is contained in one tablet.

TABLE 14

| Items | Production conditions |
|---|---|
| Punch size and | φ 8.0 mm, R: 12 mm |
| shape | |
| Rotation speed | 30 rpm |
| Main pressure | 10 kN |

R means the radius of curvature. The same applies hereinafter.

### (6) Preparation of Film Coating Liquid (3)

Purified water in an amount of 1084.20 kg was weighed put into a stainless steel tank, and sodium lauryl sulfate and stearic acid were then charged, and dispersed/dissolved. Then, amino alkyl methacrylate copolymer E was added, and dissolved/dispersed with a disperser for 1.5 hours. Then, talc was added, and further dissolved/dispersed with the disperser for 30 minutes. While stirring with the disperser, Red No. 2 was added and dissolved. The resulting liquid was allowed to pass through a screen having a mesh opening of 500 μm to obtain a film coating liquid (3).

### (7) Production of Film Coated Tablet Containing 1 mg of Haloperidol

Film coated tablets containing 1 mg of haloperidol were produced under the conditions shown in Table 15. First. 1000(140 g of uncoated tablets were charged into a coating machine (Freund Corp., model HCT-30N), and heated under the conditions of the preheating step in Table 15 while rotating a coating pan in inching motion until outlet temperature became 40° C. Subsequently, the film coating liquid (3) was sprayed under the conditions of the spray step in Table 15, and continued film coating until the coating amount became about 5.3 mg. It is noted that tablets were also sampled in the course of coating when the coating amount became about 1.3 mg. Then, drying was performed under the conditions of a temperature of 40° C. and a duration of 2 hours in a shelf dryer to produce film coated tablets each having a coating amount of film coating of about 1.3 mg or about 5.3 mg.

TABLE 15

| Items | Production conditions | | |
|---|---|---|---|
| Steps | Preheating | Spraying | Drying |
| Number of guns (pieces) | — | 1 | — |
| Spray air pressure (MPa) | — | 0.15 | — |
| Outlet temperature (° C.) | 80 | 80 | 60 |
| Charge air flow (m³/min) | 0.7 | 0.60 to 0.65 | 0.60 to 0.65 |
| Spray rate (g/min) | | about 5 | — |
| Rotation speed of pan (rpm) | Inching | 11~21 | 11 |
| | rotation | | |
| Adequate required time | 6 | 55 | 120 |
| (minutes) | | | |

### (Evaluating Coating Amount of Film Coating and Thickness of Film Coated Tablet)

With regard to Example 2 and Comparative Example 1, the coating amount of film coating of the film coated tablets was calculated from the weight of tablets, and the thickness was also measured. Results from the measurements are shown in Table 16.

### <Conditions for Measuring Thickness of Film Coating Layer>

A tablet is cut into two pieces in the middle in the direction perpendicular to the top surface of the tablet using a cutter knife, and the thickness of the film coating layer was measured under a laser microscope (VK-X100, available from Keyence Corporation) or a motion analysis microscope (VW-6000/5000, available from Keyence Corporation).

TABLE 16

| | | Shapes | Coating | | | | |
|---|---|---|---|---|---|---|---|
| | | of | Amount | Thickness of film coating | | | |
| | | uncoated | of film | Top | Under | Side | |
| | Tablet | tablet/ | coating | Sur- | sur- | sur- | Av- |
| | types | tablet | (/tablet) | face | face | face | erage |
| Ex- | Film | Round | 2.4 mg | 4.4 μm | 7 μm | 5.2 μm | 5.6 μm |
| ample | coated | tablet, | | | | | |
| 2 | tablet | 8.0 mm | | | | | |
| | | in | | | | | |
| | | diameter, | | | | | |
| | | planar | | | | | |
| | | rounded- | | | | | |
| | | corner | | | | | |

| | | | | | | |
|---|---|---|---|---|---|---|
| Com- | Film | Round | 5.3 mg | 54.8 μm* | 28.2 μm | 45.9 μm |
| para- | coated | tablet, | | | | |
| tive | tablet | 8.0 mm | 1.3 mg | 16.4 μm* | 9.4 μm | 14.1 μm |
| Ex- | | in | | | | |
| ample | | diameter, | | | | |
| 1 | | R: 12.0 | | | | |

*No distinction between the top and under surfaces in Comparative Example 1 because the film-coated tablets were prepared by performing film coating according to the pan coating method. Therefore, the same value is shown.

As clearly shown in Table 16, the film-coated tablets in Example 2 showed less variation in the thickness of film coating between the top and under surfaces and the side surfaces, as compared to Comparative Example 1. Moreover, in terms of the thickness of film coating, film-coated tablets having a thinner thickness was able to be obtained in Example 2 as compared to those produced by pan coating in Comparative Example 1.

### (Dissolution Test)

In accordance with Dissolution Test defined in General Tests of the Japanese pharmacopoeia, the film coated tablets produced in Example 2, Example 3, and

### Comparative Example 1 were Subjected to Dissolution Tests Under the Following Conditions

### <Conditions for Dissolution Tests>

Method: Dissolution Test defined in General Tests of the Japanese pharmacopoeia (paddle method)

Rotation speed: 50 rpm

Volume of test medium: 900 mL

Temperature of test medium: 3PC±0.5° C.

Test medium: a solution defined in 2nd Fluid for Dissolution Test

Number of tests: N=3

Sinker: Yes

### <Measurement of Dissolution Ratio>

A UV probe was placed in a test vessel containing 900 mL of a test medium, and a tablet was then placed in the vessel to measure an absorbance value. A dissolution ratio was calculated from the absorbance value obtained.

### <Analysis Conditions>

Instrument: Rainbow (Registered Trademark, available from Pion Inc.)

Tip: 20 mm

Detection wavelength: 248 nm

Results from the dissolution tests are shown in Tables 17 and 18.

TABLE 17

| | | | Average | Average dissolution | |
|---|---|---|---|---|---|
| | Shapes of uncoated | | thickness of | ratio (%) | |
| | tablet/tablet | Tablet types | film coating | 30 min | 60 min |
| Example 2 | Round tablet, 8.0 mm | Film coated | 5.6 μm | 5.11 | 7.61 |
| | in diameter, planar | tablet | | | |
| | rounded-comer | Uncoated tablet | | 99.74 | 104.61 |
| Comparative | Round tablet, 8.0 mm | Film coated | 45.9 μm | 4.11 | 8.66 |
| Example 1 | in diameter, R: 12.0 | tablet | 14.1 μm | 37.06 | 78.5 |
| | | Uncoated tablet | | 98.74 | 102.19 |

TABLE 18

| | | | Average | | Average | |
|---|---|---|---|---|---|---|
| | | | thick- | Num- | dissolution | |
| | Shapes of | | ness | ber | ratio (%) | |
| | uncoated | Tablet | of film | of | 30 | 60 |
| | tablet/tablet | types | coating | pores*1 | min | min |
| Example | Round tablet, 8.0 mm | Film | 5.6 μm | 1 | 1.77 | 4.18 |
| 2 | in diameter, planar | coated | | 3 | 2.99 | 6.29 |
| | rounded-corner | tablet | | 5 | 4.77 | 8.51 |

*Pores were created by leaving circular portions unprinted so that the circular portions each have a diameter of 1 mm.

As shown in Table 17, the film coated tablets from Example 2, which had an average film coating thickness of 5.6 μm, showed a dissolution ratio comparable with that of the film coated tablets (Comparative Example 1) having an average film coating thickness of 45.9 μm which were produced according to the conventional pan coating method. The film coated tablets (Comparative Example 1) having an average film coating thickness of 14.1 μm which were produced according to the common pan coating method showed a dissolution ratio of 37.06% at the time of 30 minutes. As apparent from this, the dissolution ratio was difficult to be controlled when the thickness of film coating was thin as prepared by the conventional pan coating method. These clearly state that the film coated tablets from Example 2 was able to show a reduced dissolution ratio even when the thickness of film coating was thinner than that of the film coated tablets from Comparative Example 1 which was produced according to the conventional pan coating method.

Further, Example 2 enabled an uncoated tablet having a planar rounded-corner shape to be successfully coated, and also enabled the dissolution properties to be controlled. Such a tablet was difficult to be produced by the conventional pan coating method.

Moreover, as seen from Table 18, in the case of the film coated tablets from Example 3, it was observed that the dissolution ratio of an active ingredient was able to be controlled by varying the number of pores formed in a controlled-release film itself.

1. A solid preparation comprising:
a tablet core, and
at least one layer of a controlled-release film provided on at least a part of a surface of the tablet core,
wherein the tablet core contains at least one active ingredient, and
the controlled-release film includes a dry film of an edible ink containing at least one water-insoluble polymer.

2. The solid preparation according to claim 1, wherein the at least one layer of the controlled-release film is a patterning layer.

3. The solid preparation according to claim 2, wherein patterning of the controlled-release film is different for each of any regions in a plane.

4. The solid preparation according to claim 1, wherein the at least one layer of the controlled-release film has at least one pore for releasing the active ingredient to an outside.

5. The solid preparation according to claim 4, wherein the total opening area of the pore is in a range of 0.2% and 50% relative to the surface area of the tablet core.

6. The solid preparation according to claim 1, wherein the thickness of the at least one layer of the controlled-release film varies in a plane.

7. The solid preparation according to claim 1, having a structure having multiple layers of the controlled-release film.

8. The solid preparation according to claim 1, wherein the thickness of the controlled-release film is in a range of 0.1 μm to 50 μm.

9. The solid preparation according to claim 1, wherein the controlled-release film contains one or two or more the water-insoluble polymers having a solubility of 10 g/100 g or less in water or in an aqueous solution having a pH within the range of 1.2 to 8 at 25° C.

10. The solid preparation according to claim 1, wherein the water-insoluble polymer is at least one selected from the group consisting of stomach-soluble polymers, enteric polymers, and other water-insoluble polymers.

11. The solid preparation according to claim 1, wherein the shape of the tablet core is of a flat tablet or a splitting tablet.

12. The solid preparation according to claim 1, wherein the active ingredient is thermally unstable.

13. The solid preparation according to claim 1, wherein the controlled-release film includes a dry film of the edible ink having a viscosity in a range of 1 mPa·s to 1,000,000 mPa·s.

14. The solid preparation according to claim 1, wherein the controlled-release film controls the release of the active ingredient contained in the tablet core.
